(19) United States
(12) Patent Application Publication
(10) Pub. No.: US 2004/0115654 A1
(43) Pub. Date: Jun. 17, 2004

(54) Laser exposure of photosensitive masks for DNA microarray fabrication

(75) Inventors: Valluri Rao, Saratoga, CA (US); Mineo Yamakawa, Campbell, CA (US); Andrew A. Berlin, San Jose, CA (US)

Correspondence Address: SCHWEGMAN, LUNDBERG, WOESSNER & KLUTH, P.A., P.O. BOX 2938, MINNEAPOLIS, MN, 55402, US

(73) Assignee: Intel Corporation

(21) Appl. No.: 10/320,041

(22) Filed: Dec. 16, 2002

(51) Int. Cl. C12Q 1/68; B05D 3/00; C12M 1/34

(52) U.S. Cl. 435/6; 435/287.2; 427/2.11

(57) ABSTRACT

A method and apparatus for forming a polymer array on a substrate suitable for synthesizing polymer sequences. This includes forming an array, each location of the array having at least one strand end, forming photosensitive protection on the strand ends, and selectively scanning and modulating at least one energy beam to expose a pattern on the photosensitive protection. In some embodiments, the method further includes removing a protective group from selected strand ends based on the exposed pattern. The method then includes adding a predetermined one or more polymeric subunits to the deprotected strand ends. In some embodiments the photosensitive protection includes a layer of photoresist to cover the strand ends. Some embodiments use an ultra-violet laser.

### FIELD OF THE INVENTION

This invention relates to the field of polymer synthesis, and more specifically to a method and apparatus of making a biological DNA-tag microarray.

### BACKGROUND OF THE INVENTION

DNA, RNA, and other oligonucleotides (as well as other polymers) can be synthesized by performing a series of individual chemical reactions. Some biological materials, such as RNA or DNA, are polymers of oligonucleotides, such as A, C, G, and T. RNA, DNA, and other polymers such as glycoproteins can be synthesized with any desired sequence by depositing an initial molecule onto a surface of a substrate, activating an end of the molecule, and then washing the surface with a solution containing a large number of the next desired amino group, that, once attached to the free end of the initial molecule, will also deactivate that end. Thus, since the activated end of the strand is deactivated by the attachment of the next amino group, only a single instance of that desired amino group will attach at each step of the process.

By starting with a large number of initial molecules attached to one area of the substrate, a large number of identical sequence strands can be made in parallel. For example, a large number of identical DNA sequences can be formed by starting with a large number of primer entities attached to a solid surface (such as a glass or silicon substrate), and then adding one nucleotide or amino group at a time to form the desired sequence on every primer. Such a process will provide a large number of oligonucleotides each identical to the rest.

Often, it is also desired to have a plurality of different sequences on a chip (typically a small rectangular substrate), one type of sequence per area or location of the chip. For example, the chip can be laid out as an array of rows and columns, with each row-and-column address defining a location for a synthesized polymer. A large number of chips can be made in parallel on a single substrate, which is then diced into individual chips. Polymer arrays and so-called DNA chips typically include many individual sites onto which a series of monomers or partial DNA sequences are sequentially attached. At each predefined location of the array, one or more identical sequences will be formed (depending on the number or primer sited provided at that location), but different locations of the array will typically each have a different sequence. Photolithographic processes can be used to define the order of the sequence being built at each location by selectively activating or deactivating each array location from participation in each successive step. For example, chrome-on-glass masks can be used to define a pattern on photoresist that defines which array locations are activated for each attachment step. Manufacturing such arrays in parallel can provide a large number of identical arrays, where each element of each array is different than other elements of the same array.

There is a need for an improved method and apparatus that provides accuracy, flexibility and speed/performance in defining the active array locations for each nucleotide-adding operation.

### BRIEF DESCRIPTION OF THE DRAWINGS

FIG. 1 shows a block diagram system 100 for making arrays of predetermined sequences

FIG. 2 shows a flow chart of a method 200 according to the invention.

FIG. 3 is an isometric view of a substrate 99.

FIG. 4 is a cross section view of substrate 99.

FIG. 5 is an enlarged cross section view of a well 95 on substrate 99.

### DESCRIPTION OF PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention.

The leading digit(s) of reference numbers appearing in the Figures generally corresponds to the Figure number in which that component is first introduced, such that the same reference number is used throughout to refer to an identical component which appears in multiple Figures. The same reference number or label may refer to signals and connections, and the actual meaning will be clear from its use in the context of the description.

Some biological materials, such as RNA or DNA, are polymers of oligonucleotides, such as A, C, G, and T. RNA, DNA, peptides of amino acids or analogs thereof, and other polymers such as glycoproteins can be synthesized with any desired sequence by depositing an initial molecule or primer onto a surface of a substrate or insoluble matrix. The substrate is, e.g., made of glass, silicon, plastics or other polymers, e.g., polystyrene, polypropylene, etc., selected to be compatible with the full range of conditions to which the device will be exposed, e.g., extremes of temperature, salt, or pH, application of electric fields, e.g., in electrophoretic analysis embodiments, as well as compatibility with reagents and other materials used in fabricating the devices.

In some embodiments, amino acids are added stepwise to a growing peptide chain that is linked to the insoluble matrix or substrate using a solid-phase method devised by R. Bruce Merrifield and well known in the art. The carboxyl-terminal amino acid of the desired peptide is first anchored to the substrate. The t-Boc protecting group of this amino acid is then removed. The next amino acid (in the protected t-Boc form) is then added together with dicyclohexylcarbodiimide, which is the coupling agent. After formation if the peptide bond, excess reagents and dicyclohexylurea are washed away, which leaves the substrate with the desired initial chain. Further amino acids are added by repeating the above sequence of reactions.

Similarly, in some embodiments, DNA strands are synthesized by the sequential addition of activated monomers to a growing chain linked to an insoluble substrate. In some embodiments, the activated monomers are protonated deoxyribonucleoside 3′phosporamidites. The 3′-phosphorus atom of the incoming unit becomes joined to the 5′-oxygen of the growing chain to form a phosphite triester. The 5′OH of the activated monomer is unreactive to further reactions because it is blocked by a dimethoxytrityl (DMT) protecting group. In some embodiments, coupling is carried out under anhydrous conditions because water reacts with phosphoramidites. Next, a phosphite triester (in which P is trivalent) is oxidized by iodine to form a phosphotriester (in which P is pentavalent). In a third step, the DMT protecting group is removed by the addition of dichloroacetic acid, which leaves other protecting groups intact. The growing DNA chain is now one unit longer and ready for another cycle of addition as described above. This solid-phase process is desirable, as with peptides described above, because the desired strand stays in place on the substrate.

In some embodiments, one method of the present invention covers the substrate with photoresist, a light activated chemical that does not chemically participate in the synthesis of the desired (e.g., oligonucleotide or peptide) chain, but which provides a barrier over selected portions of the substrate during one step such that only the uncovered portions participate in the synthesis reactions. Exposure to a pattern of light (e.g., a scanned UV laser beam) allows removal of portions of the photoresist in a predetermined pattern. The substrate is then processed to activate an end of the uncovered molecules, and then provided a solution having many copies of a single amino group, for example, a single A, C, G, or T with the appropriate protecting group such that one and only one copy of the desired nucleotide or amino acid is attached to every activated primer location. Once the appropriate conditions (e.g., time, acidity, temperature, concentration, etc.) have been provided to attach the desired one group to each primer location, the solution is washed away, and the substrate is prepared for the next attachment step, by e.g., again activating the ends of the partially built strands. The next step would use a different pattern of light such that different sequences are built at each of a plurality of sites on the array.

In some embodiments, by covering the substrate with a photoresist at the start of each step, an array of locations can be defined, such that a different sequence can be fabricated at each array location. As used herein, an “array” is any pattern having a plurality of positions, such as a linear pattern, a pattern of rows and columns, a hexagonal pattern or any other pattern of positions (wherein such positions are also called array locations). Some conventional methods use a photolithographic mask, e.g., a conventional chrome-on-glass mask, to define which locations are activated and which are not activated for each attachment step. Other conventional methods use a micro-electro-mechanical system (MEMS device) having an array of tiny mirrors, where some of the mirrors are tilted (“on”) to direct light in the desired activation pattern on the substrate, and the other mirrors are differently tilted (“off”) to direct light elsewhere. In either the mask case or the MEMS case, an extremely bright UV light source is needed that has optical properties that are expensive to achieve. Further, to fabricate a ten-million-well microarray, ten million functional mirrors are required. Typically, a computer program is used to define the series of masks to be used, and each mask is individually made with its own pattern, and then each mask is used in one step of the synthesis process. To change the set of sequences on the microarray, a new set of masks must be generated.

In other embodiments, rather than use a physical photoresist barrier to either cover or expose the underlying chains to a particular step of the chain building described above, a photo-activated protective group is used as each monomer is added. Thus, rather than using a strictly chemical removal of the protecting group in areas not covered by the photoresist barrier, no photoresist barrier is used but instead the protective group itself is sensitive to light and is removed after selective exposure to light of the array locations that are to be included in that step of chain building.

To make an arbitrary polymer sequence having four oligonucleotides at each of twenty-five locations (i.e., each polymer strand having a length of twenty five), a simple process would use possibly four different masks for each location, and thus up to 100 different masks.

In some embodiments, the process is modified to use a direct-write scanning process to define the pattern of activation and deactivation for each step, wherein a light source (such as an ultraviolet laser) or an electron beam is scanned over the photoresist surface (or, in other embodiments, onto the photo-activated protective groups), exposing or not exposing each array location to define the pattern of activation and deactivation for the next step. A computer program is used to define the pattern and control the exposure to obtain the desired monomer. During the scan, the light or electron beam is directed onto each of a plurality of predetermined locations on a surface (for example, an XY Cartesian array), and then modulated on or off to selectively activate certain ones of the array locations.

In some embodiments, the present invention also provides a method for fabricating a plurality of nucleic acid array chips on a substrate, dicing or otherwise separating the individual arrays, and packaging the resulting chips. Some embodiments contemplate, and have incorporated the use of photoresistive plastics. In some embodiments, the devices of the invention are made using microfabrication techniques generally used in the semiconductor and microelectronics industries. These techniques include film deposition processes such as spin coating, electrodeposition, low-pressure vapor deposition, laser fabrication processes, photolithographic methods such as UV or X-ray or e-beam processes, and/or etching processes which may be performed by either wet chemical processes or plasma processes.

Herein, planar members are also referred to herein as “substrates,” “slides” or “chips.” These planar members are, in various embodiments, made from a variety of materials, including, e.g., plastics (compression molded, injection molded, machined, etc.), glass, silicon, quartz, or other silica-based materials, and the like. In some embodiments, the substrate includes a plurality of depressions, wells or cavities on the surface. Each cavity forms the basis of a reaction chamber, and is generally disposed within the first planar member, or may be a combination of an aperture in the first and a well region or indentation in the second. This cavity may be machined or etched into the surface of the first planar member, or alternative, may be prepared in the manufacturing of the first planar member, such as where the planar member is a molded part, e.g., plastic.

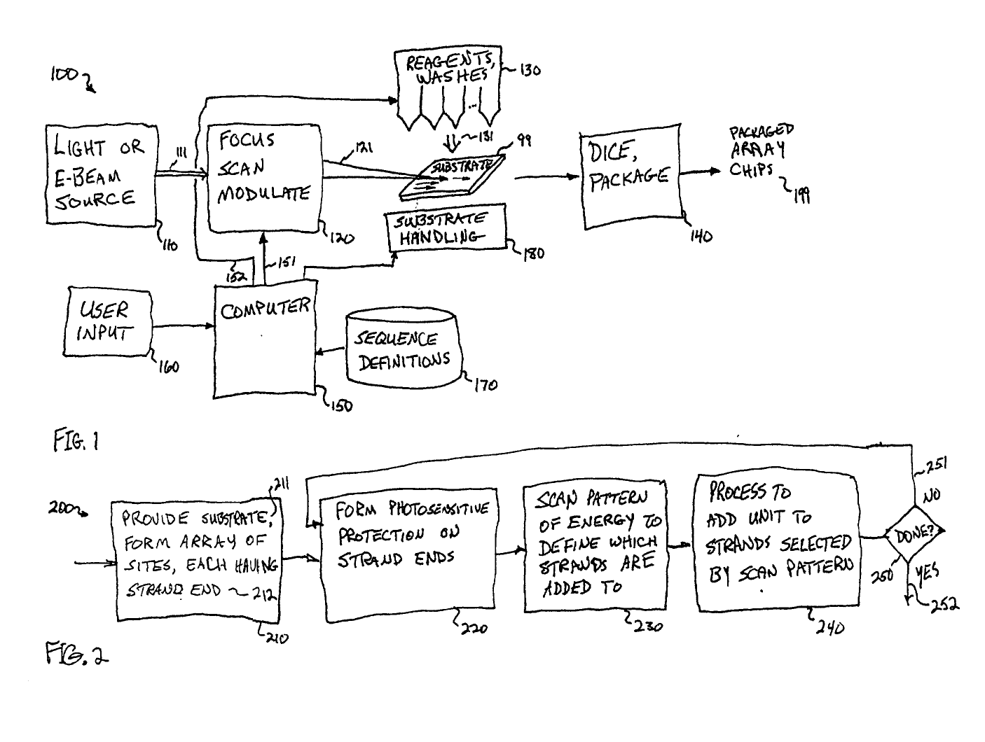

FIG. 1 shows one embodiment of the present invention having a system 100 for making arrays of predetermined sequences. In some embodiments, the arrays 199 are of DNA strands. In other embodiments, the arrays 199 are of RNA strands. In yet other embodiments, the arrays 199 are of peptides. In still other embodiments, the arrays 199 are of other polymer sequences. A light or e-beam source 110 (such as, e.g., an ultraviolet laser) provides a beam 111 (of light or electrons) that is focused, scanned, and/or modulated by device 120 under the control of signals 151 from computer 150. In various embodiments, computer 150 is implemented as any suitable controlling entity, such as any information processing system, personal computer, microprocessor, programmable logic array, microcontroller, and/or internet-connected workstation; and the term “computer” is meant to include any such entity. The resulting beam 121 is scanned across substrate 99 to define the locations that are to participate in the next reaction. In other embodiments, a plurality of ultraviolet lasers 110 and a plurality of devices 120 are used to modulate and scan a plurality of beams 121 simultaneously, in order to speed throughput (i.e., reduce the time per step).

In some embodiments, signals 152 from computer 150 control the dispensing of reagents and washes 131 from dispensers 130, in order to add each successive monomer (such as an oligonucleotide or amino acid). For example in a DNA synthesis, if an A nucleotide is to be added, the computer 150 would direct device 120 to expose all areas to which an A is the next desired nucleotide, and the substrate is then processed to activate the strand ends in those areas (e.g., by removing a protective group). The A nucleotide with its protective group is then added to all the strand ends, wherein the protective group on the A nucleotide prevents more than one A being added during this step. Next, if a C nucleotide is to be added, the computer 150 would direct device 120 to expose all areas to which a C is the next desired nucleotide, and the substrate is then processed to activate the strand ends in those areas. The C nucleotide with its protective group is then added to all the strand ends. Thus, in successively repeated steps controlled by computer 150, the desired set of various DNA strands are simultaneously built in an array pattern on substrate 99.

In some embodiments, signals 153 from computer 150 control substrate handling device 180 to perform such operations such as moving the substrate when required, and/or controlling temperature and other environmental factors as needed for the reactions. In some embodiments, apparatus 180 includes a spin deposition station that deposits a suitable amount of photoresist and then spins the substrate to form a thin uniform layer of photoresist over the substrate, as is well known in the integrated circuit art.

In some embodiments, once all the desired strands have been built, the substrate 99 is diced and packaged using device 140 to provide the finished packaged array chips 199.

In some embodiments, computer 150 receives user input to define the desired arrays using sequence definitions stored in storage 170. Because the patterns are directly written onto substrate 99 using beam 121, no mask generation steps are needed, and thus more flexibility and faster turnaround times can be achieved. Further, in some embodiments, computer 150 looks ahead at various sequences that could be used to generate the desired set of sequences, and optimizes the order of nucleotide, attachment. For example, rather than repeating the steps for A, C, G, and T attachment twenty-five times (a total of one hundred steps) to generate a plurality of oligonucleotides each twenty-five units long, computer 150 reorders the steps to achieve fewer overall steps, for example, by sequencing A, C, C, T, G, . . . if such a sequence required fewer steps to complete the set of oligonucleotides.

The present invention eliminates the need for repetitive mask steps that plague conventional fabrication methods without introducing the need to fabricate a huge array of MEMS micro-mirrors. The present approach is more reliable, scaleable and inexpensive than conventional approaches.

The present invention provides methods and apparatus for the preparation and use of a substrate having a plurality of polymer sequences in predefined regions. Such are further described in U.S. Pat. No. 5,445,934 to Fodor et al. The invention is described herein primarily with regard to the preparation of molecules containing sequences of nucleotides or amino acids, but could readily be applied in the preparation of other polymers. Such polymers include, for example, both linear and cyclic polymers of nucleic acids, glycoproteins, polysaccharides, phospholipids, and peptides having either .alpha.-, beta.-, or .omega.-amino acids, heteropolymers in which a known drug is covalently bound to any of the above, polyurethanes, polyesters, polycarbonates, polyureas, polyamides, polyethyleneimines, polyarylene sulfides, polysiloxanes, polyimides, polyacetates, or other polymers which will be apparent upon review of this disclosure. In some embodiments, the invention herein is used in the synthesis of peptides.

The prepared substrate may, for example, be used in screening a variety of polymers as ligands for binding with a receptor, although it will be apparent that the invention could be used for the synthesis of a receptor for binding with a ligand. The substrate disclosed herein will have a wide variety of other uses. For example, the invention herein can be used in determining peptide and nucleic acid sequences that bind to proteins, finding sequence-specific binding drugs, identifying epitopes recognized by antibodies, and evaluation of a variety of drugs for clinical and diagnostic applications, as well as combinations of the above.

In some embodiments, linker molecules are provided on a substrate. A terminal end of the linker molecules is provided with a reactive functional group protected with a photoremovable protective group. Using lithographic methods, the photoremovable protective group is exposed to light and removed from the linker molecules in first selected regions. The substrate is then washed or otherwise contacted with a first monomer that reacts with exposed functional groups on the linker molecules. In some embodiments, the monomer is an amino acid containing a photoremovable protective group at its amino or carboxy terminus and the linker molecule terminates in an amino or carboxy acid group bearing a photoremovable protective group. In other embodiments, the monomer is a nucleotide containing a photoremovable protective group at its terminus.

A second set of selected regions is, thereafter, exposed to light and the photoremovable protective group on the linker molecule/protected amino acid is removed at the second set of regions. The substrate is then contacted with a second monomer containing a photoremovable protective group for reaction with exposed functional groups. This process is repeated to selectively apply monomers until polymers of a desired length and desired chemical sequence are obtained. Photolabile groups are then optionally removed and the sequence is, thereafter, optionally capped. Side chain protective groups, if present, are also removed.

By using the lithographic techniques disclosed herein, it is possible to direct light to relatively small and precisely known locations on the substrate. It is, therefore, possible to synthesize polymers of a known chemical sequence at known locations on the substrate.

The invention preferably provides for the use of a substrate 99 having a well 95 with a surface “S” (see FIG. 5). Linker molecules “L” are optionally provided on a surface of the substrate. The purpose of the linker molecules, in some embodiments, is to facilitate receptor recognition of the synthesized polymers.

Optionally, the linker molecules may be chemically protected for storage purposes. A chemical storage protective group such as t-BOC (t-butoxycarbonyl) may be used in some embodiments. Such chemical protective groups would be chemically removed upon exposure to, for example, acidic solution and would serve to protect the surface during storage and be removed prior to polymer preparation.

On the substrate surface S or on a distal end of the linker molecules, a functional group with a protective group 97 is provided. The protective group 97 may be removed upon exposure to radiation, electric fields, electric currents, or other activators to expose the functional group.

In some embodiments, the radiation is ultraviolet (UV), infrared (IR), or visible light. As more fully described below, the protective group may alternatively be an electrochemically-sensitive group, which may be removed in the presence of an electric field. In still further alternative embodiments, ion beams, electron beams, or the like may be used for deprotection.

In some embodiments, the exposed regions and, therefore, the area upon which each distinct polymer sequence is synthesized are smaller than about one mm square. In some embodiments the exposed area is less than about 10,000 microns (micrometers) square or, in other embodiments, less than one hundred microns square, less than 10 microns square, or less than one micron square, and may, in some embodiments, encompass the binding site for as few as a single molecule. Within these regions, each polymer is preferably synthesized in a substantially pure form (all sequences being substantially the same).

One aspect of the present invention provides a method 200, such as shown in FIG. 2, for forming a polymer array, the method including providing 211 a substrate suitable for synthesizing polymer sequences, forming 212 an array, each location of the array having at least one strand end, forming 220 photosensitive protection on the strand ends, and selectively scanning and modulating 230 at least one energy beam to expose a pattern on the photosensitive protection. In some embodiments, the method further includes removing a protective group from selected strand ends based on the exposed pattern. The method then includes adding 240 a predetermined one or more units to the deprotected strand ends.

In some embodiments of the method, the forming photosensitive protection includes depositing a layer of photoresist to cover the strand ends. In some embodiments of the method, the one or more energy beams include light from an ultra-violet laser. In some embodiments of the method, the polymer sequences are DNA sequences. In some embodiments of the method, the substrate includes a plurality of wells formed on a major surface of the substrate. In some embodiments of the method, the selectively scanning and modulating includes simultaneously scanning two or more beams of light from ultraviolet lasers.

FIG. 3 is an isometric schematic (not to scale) view of a substrate 99 having a photoresist layer 91 on a base 90. Photoresist layer 91 is exposed to light or other scanned focused energy beam in a pattern determined by computer 150 (see FIG. 1), and processed to form openings 93.

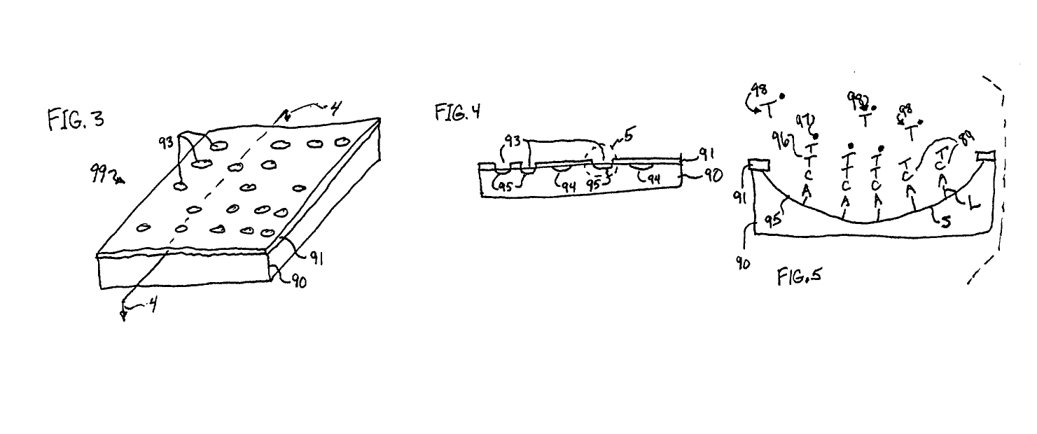

FIG. 4 is a cross section view of substrate 99. In some embodiments, substrate 99 has a plurality of wells, some of the wells 95 are exposed by openings 93, and others of the wells 94 remain covered by the photoresist later 91.

In other embodiments, wells are not used, but rather the top surface of the base 90 is substantially flat, and the polymer strands are built on that flat top surface, with the photoresist layer 91 deposited directly on the growing strands, exposed to a modulated, scanned, and focused energy beam (such as an ultraviolet laser light beam) in a pattern determined by computer 150 (see FIG. 1), and processed to form openings 93. In some embodiments, a negative photoresist is used, such that the modulated, scanned, and focused ultraviolet laser beam is turned on where holes in the photoresist are desired to soften the photoresist, and a solvent is used to remove the softened photoresist. In other embodiments, a positive photoresist is used, and the unexposed portions of the photoresist are removed to form the holes 93.

FIG. 5 is an enlarged cross-section view of a well 95 on substrate 99. In the embodiment shown, a plurality of DNA strands have been formed, each being a sequence (e.g., -ACT) that is attached to a surface S of the substrate 99 using a linking unit L. In this example, the hole 93 allows into the well 95 a solvent having the next unit being attached T, shown with a protective group indicated by the dot 97. Three of the sequences (labeled 96) have already attached this unit and its protective group, while two of the sequences (labeled 89) are still activated (as indicated by no protective group 97). The free T-dot groups 98 will soon attach to the unprotected ends of sequences 89.

In some embodiments, this method includes the following operations:

deposit an appropriate amount of liquid photoresist on the surface of base 90, and spin the base to distribute the photoresist into a thin layer 91;

scan a modulated pattern of light 121;

develop the photoresist to form openings 93 at locations to which the next monomer unit is to be attached;

remove the protective groups at the end of all strands that are exposed by the openings 93;

flood the surface with a liquid containing many copies of the next monomer 98, each copy having a protective group 97 that stops the chain building once one copy is attached to the strands 96 on the substrate;

rinse and repeat these operations.

In other embodiments, a shortened method is used in which the protective group itself is photosensitive, thus removing the need for the photoresist operations. This method includes the following operations:

start with strands, each attached to base 90 at their attached ends, that each have a photosensitive protective group at their respective free ends;

scan a modulated pattern of light 121, and remove the photosensitive protective groups at the end of all strands that were exposed to the light;

flood the surface with a liquid containing many copies of the next monomer 98, each copy having a photosensitive protective group 97 that stops the chain building once one copy is attached to the strands 96 on the substrate 99;

rinse and repeat these operations.

### CONCLUSION

One aspect of the present invention provides a method 200, such as shown in FIG. 2, for forming a polymer array, the method including providing 211 a substrate suitable for synthesizing polymer sequences, forming 212 an array, each location of the array having at least one strand end, forming 220 photosensitive protection on the strand ends, and selectively scanning and modulating 230 at least one energy beam to expose a pattern on the photosensitive protection. In some embodiments, the method further includes removing a protective group from selected strand ends based on the exposed pattern. The method then includes adding 240 a predetermined one or more polymeric subunits to the deprotected strand ends.

Another aspect of the present invention provides a system 100 (see FIG. 1) for forming a polymer array on a substrate. System 100 includes an energy-beam source 110 that produces an energy beam, a scanner 120 configured to move the energy beam in a scanned pattern on the substrate, a computer 150 operatively coupled to the scanner to control the scanned pattern of energy according to a sequence definition for each of a plurality of desired polymer sequences being synthesized, a reagent source 130 configured to add one of a plurality of monomers at each end location of each of a plurality of strands formed on the substrate, and apparatus 180 to provide photosensitive protection that controls an order of monomers based on the scanned pattern of light.

In some embodiments, the apparatus 180 includes apparatus that deposits a layer of photoresist 91 to cover the strand ends.

In some embodiments, the energy-beam source includes an ultra-violet laser.

In some embodiments, the polymer sequences are DNA sequences 96.

In some embodiments, the substrate includes a plurality of wells 95 formed on a major surface of the substrate 99.

In some embodiments, the energy-beam source 110 produces two or more beams of light from ultraviolet lasers.

Some embodiments further include a modulator, operatively coupled to be controlled by the computer, that modulates an intensity of the light.

Still another aspect of the present invention provides a system 100 (see FIG. 1) that includes a first ultraviolet laser 110, a first scanner 120 configured to move light from the first ultraviolet laser in a scanned pattern on the substrate, a computer 150 operatively coupled to the first scanner to control the scanned pattern of light according to a sequence definition for each of a plurality of desired polymer sequences 96 being synthesized, a reagent source 130 configured to add one of a plurality of monomers at each end location of each of a plurality of strands formed on the substrate; and apparatus 180 to provide photoresist layer that is patterned by the scanned light pattern to controls an order of monomers based on the computer-controlled scanned pattern of light.

Some embodiment further include a second ultraviolet laser 110, and a second scanner 120 configured to move light from the second ultraviolet laser in a scanned pattern on the substrate, wherein the computer 150 is operatively coupled to the second scanner 120 to control the scanned pattern of light according to a sequence definition for each of a plurality of desired polymer sequences being synthesized.

In various embodiments of the invention, different sets of polymeric subunits are used as reagents in building the desired polymer strand with regard to the preparation of molecules containing sequences of nucleotides or amino acids, but could readily be applied in the preparation of other polymers. Such polymers include, for example, both linear and cyclic polymers of nucleic acids, polypeptides, polysaccharides, phospholipids, and peptides having either alpha.-, beta.-, or omega.-amino acids, heteropolymers in which a known drug is covalently bound to any of the above, polyurethanes, polyesters, polycarbonates, polyureas, polyamides, polyethyleneimines, polyarylene sulfides, polysiloxanes, polyimides, polyacetates, or other polymers which are apparent upon review of this disclosure. The term “polymeric subunits” means one or more of the basic subunits that are put together to form the desired polymer.

It is understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled

What is claimed is:

1. A method for forming a polymer array, the method comprising:
providing a substrate suitable for synthesizing polymer sequences;
forming an array, each one of a plurality of locations of the array having at least one strand end;
forming photosensitive protection on the strand ends;
selectively scanning and modulating at least one energy beam to expose a pattern on the photosensitive protection;
removing a protective group from selected strand ends based on the exposed pattern; and
adding a predetermined one or more polymeric subunits to the deprotected strand ends.

2. The method according to claim 1, wherein the forming photosensitive protection includes depositing a layer of photoresist to cover the strand ends.

3. The method according to claim 1, wherein the one or more energy beams includes light from an ultra-violet laser.

4. The method according to claim 1, wherein the polymer sequences are DNA sequences.

5. The method according to claim 1, wherein the substrate includes a plurality of wells formed on a major surface of the substrate.

6. The method according to claim 1, wherein the selectively scanning and modulating includes simultaneously scanning two or more beams of light from ultraviolet lasers.

7. A system for forming a polymer array on a substrate, the system comprising:
an energy-beam source that produces an energy beam;
a scanner configured to move the energy beam in a scanned pattern on the substrate;
a computer operatively coupled to the scanner to control the scanned pattern of energy according to a sequence definition for each of a plurality of desired polymer sequences being synthesized;
a reagent source configured to add one of a plurality of monomers at each end location of each of a plurality of strands formed on the substrate; and
apparatus to provide photosensitive protection that controls an order of monomers based on the scanned pattern of light.

8. The system according to claim 7, wherein the apparatus to provide photosensitive protection includes apparatus that deposits a layer of photoresist to cover the strand ends.

9. The system according to claim 7, wherein the energy-beam source includes an ultra-violet laser.

10. The system according to claim 7, wherein the polymer sequences are DNA sequences.

11. The system according to claim 7, wherein the substrate includes a plurality of wells formed on a major surface of the substrate.

12. The system according to claim 7, wherein the energy-beam source produces two or more beams of light from ultraviolet lasers.

13. The system according to claim 14, further comprising a modulator, operatively coupled to be controlled by the computer, that modulates an intensity of the light.

14. A system for forming a polymer array on a substrate, the system comprising:
means for scanning a pattern of energy on the substrate according to a sequence definition for each of a plurality of desired polymer sequences being synthesized on the substrate;
a reagent source configured to add one of a plurality of monomers at each end location of each of a plurality of strands formed on the substrate; and
means for providing photosensitive protection that controls an order of monomers based on the scanned pattern of light.

15. The system according to claim 14, wherein the apparatus to provide photosensitive protection includes apparatus that deposits a layer of photoresist to cover the strand ends.

16. The system according to claim 14, wherein the means for scanning includes an ultra-violet laser.

17. The system according to claim 14, wherein the polymer sequences are DNA sequences.

18. The system according to claim 14, wherein the means for scanning includes two or more beams of light from ultraviolet lasers.

19. The system according to claim 14, wherein the means for scanning includes a modulator for modulating an intensity of the light.

20. A system for forming a polymer microarray on a substrate, the system comprising:
a first ultraviolet laser;
a first scanner configured to move light from the first ultraviolet laser in a scanned pattern on the substrate;
a computer operatively coupled to the first scanner to control the scanned pattern of light according to a sequence definition for each of a plurality of desired polymer sequences being synthesized;
a reagent source configured to add one of a plurality of monomers at each end location of each of a plurality of strands formed on the substrate; and
apparatus to provide photoresist layer that is patterned by the scanned light pattern to controls an order of monomers based on the computer-controlled scanned pattern of light.

21. The system according to claim 20, wherein the polymer sequences are DNA sequences.

22. The system according to claim 20, wherein the substrate includes a plurality of wells formed on a major surface of the substrate.

23. The system according to claim 20, further comprising:
a second ultraviolet laser; and
a second scanner configured to move light from the second ultraviolet laser in a scanned pattern on the substrate, wherein the computer is operatively coupled to the second scanner to control the scanned pattern of light according to a sequence definition for each of a plurality of desired polymer sequences being synthesized.
